(19) United States
(12) Patent Application Publication
(10) Pub. No.: US 2003/0153548 A1
(43) Pub. Date: Aug. 14, 2003

(54) Histamine-3 receptor ligands for diabetic conditions

(76) Inventors: Arthur A. Hancock, Libertyville, IL (US); Eugene N. Bush, Libertyville, IL (US); Marlon D. Cowart, Round Lake Beach, IL (US); Peer B. Jacobson, Libertyville, IL (US); Terry J. Opgenorth, Racine, WI (US); Youssef Bennani, Shaker Heights, OH (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/326,546

(22) Filed: Dec. 23, 2002

Related U.S. Application Data
(63) Continuation-in-part of application No. 10/043,848, filed on Jan. 11, 2002.

(51) Int. Cl. A61K 31/53; A61K 31/54; A61K 31/495; A61K 31/435; A61K 31/537; A61K 31/55

(52) U.S. Cl. 514/210.2; 514/252.01; 514/255.05; 514/256; 514/408; 514/422; 514/217.12; 514/252.12; 514/227.5; 514/237.8; 514/317; 514/337

(57) ABSTRACT

The invention relates to a method of treating a diabetic condition by administering a therapeutically effective amount of a histamine-3 receptor antagonist, including benzofuran and benzopyran derivatives of formula (I), aminoalkoxybiphenylcarboxamide compounds of formula (III), and aminoetherbiphenyl compounds of formula (IV) as described herein.

This application is a continuation-in-part of U.S. patent application Ser. No. 10/043,848, filed Jan. 11, 2002, which is hereby incorporated by reference.

### TECHNICAL FIELD

The invention relates to a new use for compounds exhibiting histamine-3 receptor activity and compositions comprising such compounds for the treatment of diabetes and diabetes-related conditions.

### BACKGROUND OF THE INVENTION

Histamine is a well-known mediator in hypersensitive reactions (e.g. allergies, hay fever, and asthma) which are commonly treated with antagonists of histamine or “antihistamines.” It has also been established that histamine receptors exist in at least two distinct types, referred to as H₁ and H₂ receptors.

A third histamine receptor (H₃ receptor) is believed to play a role in neurotransmission in the central nervous system, where the H₃ receptor is thought to be disposed presynaptically on histaminergic nerve endings (Nature 302:832-837 (1983)). The existence of the H₃ receptor has been confirmed by the development of selective H₃ receptor agonists and antagonists (Nature 327:117-123 (1987)) and has subsequently been shown to regulate the release of other neurotransmitters in both the central nervous system and peripheral organs, particularly the lungs, cardiovascular system and gastrointestinal tract.

A number of compounds exhibiting H₃ receptor activity have been reported. For example, aminoalkoxybiphenylcarboxamide compounds are described in U.S. Pat. No. 6,316,475, issued Nov. 13, 2001. International Publication WO 02/06223, published Jan. 24, 2002, and U.S. Publication 2002-0137931-A1, published Sep. 26, 2002, describe aminoetherbiphenyl compounds having H₃ receptor activity. International Publication WO 02/074758, published Sep. 26, 2002, describes benzofuran compounds having H₃ receptor activity. Such compounds have been described as histamine-3 receptor ligands.

A number of diseases or conditions may be treated with histamine-3 receptor ligands, wherein the H₃ ligand may be an antagonist, agonist or partial agonist. Such diseases or conditions include cardiovascular disorders such as acute myocardial infarction; memory processes, dementia and cognition disorders such as Alzheimer's disease and attention-deficit hyperactivity disorder; neurological disorders such as Parkinson's disease, schizophrenia, depression, epilepsy, and seizures or convulsions; cancer such as cutaneous carcinoma, medullary thyroid carcinoma and melanoma; respiratory disorders such as asthma; sleep disorders such as narcolepsy; vestibular dysfunction such as Meniere's disease; gastrointestinal disorders, inflammation, migraine, motion sickness, obesity, pain, and septic shock.

The role of H₃ receptor antagonists have been evaluated for any effect on obesity. (See, Leurs et al., Trends in Pharm. Sci. 19:177-183 (1998); Owens et al., Obes Res. 8(4):287-293 (2000); and Roberts et al., Hypertension 37(5):1323 (2001)). However, the use of H₃ receptor antagonists for diabetes or diabetes-related conditions has not yet specifically been described.

### SUMMARY OF THE INVENTION

The invention relates to a method of treating a diabetic condition comprising administering a therapeutically effective amount of a histamine-3 receptor antagonist, including benzofuran and benzopyran compounds of formula (I), aminoalkoxybiphenylcarboxamide compounds of formula (III), and aminoetherbiphenyl compounds of formula (IV) as described herein.

### DETAILED DESCRIPTION OF THE INVENTION

In its principle embodiment, the invention relates to a method of treating histamine-3 receptor mediated disorders including, for example, diabetes and diabetes-related conditions. Such diabetic conditions include, but are not limited to, type II diabetes, insulin resistance syndrome, metabolic syndrome, Syndrome X, polycystic ovary syndrome, and other associated diseases. The method is accomplished by administering a therapeutically effective amount of histamine-3 receptor antagonist compound, or a composition comprising the same, to a patient in need of such treatment. Diabetes and diabetes-related conditions may be improved by the administration of the desired compounds. Compounds suitable for the method of the invention include, but are not limited to, benzofuran, benzopyran, and aminoalkoxybiphenylcarboxamide compounds.

### Compounds for the Method of the Invention and their Preparation

Suitable benzofuran and benzopyran derivatives have the formula (I):

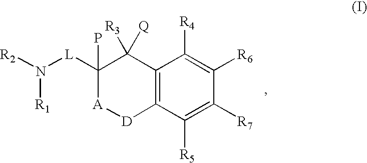

or are pharmaceutically acceptable salts, esters, amides, or prodrugs thereof, wherein

A is selected from the group consisting of carbonyl and a covalent bond;

D is selected from the group consisting of O and S;

L is selected from the group consisting of lower alkylene, fluoroalkylene, and hydroxyalkylene;

P and Q taken together form a covalent bond or are both hydrogen;

R₁ and R₂ are each independently selected from the group consisting of hydrogen, alkyl, aryl, arylalkyl, cycloalkyl, cycloalkylalkyl, heterocycle, heterocyclealkyl, hydroxyalkyl, alkenyl, and alkynyl; or

R₁ and R₂ taken together with the nitrogen atom to which they are attached, together form a heterocycle;

R₃ is selected from the group consisting of hydrogen, alkoxy, alkoxycarbonyl, alkyl, alkylcarbonyl, alkylcarbonyloxy, alkylsulfinyl, alkylsulfonyl, alkylthio, aryl, carboxy, carboxyalkyl, cyano, cyanoalkyl, formyl, halogen, haloalkoxy, haloalkyl, heterocycle, hydroxy, hydroxyalkyl, mercapto, nitro, —NR_{A}R_{B}, (NR_{A}R_{B})alkyl, (NR_{A}R_{B})carbonyl, and (NR_{A}R_{B})sulfonyl;

R₄, R₅, R₆ and R₇ are each independently selected from the group consisting of hydrogen, alkoxy, alkoxycarbonyl, alkyl, alkylcarbonyl, alkylcarbonyloxy, alkylsulfinyl, alkylsulfonyl, alkylthio, aryl, carboxy, carboxyalkyl, cyano, cyanoalkyl, cycloalkyl, formyl, halogen, haloalkoxy, haloalkyl, heterocycle, hydroxy, hydroxyalkyl, mercapto, nitro, —NR_{A}R_{B}, (NR_{A}R_{B})alkyl, (NR_{A}R_{B})carbonyl, (NR_{A}R_{B})sulfonyl, -L₂R₂₀, and —R₂₀L₃R₂₂;

L₂ is selected from the group consisting of alkylene, alkenylene, O, S, S(O), S(O)₂, C(═O), C═(NOR₂₁), and N(R_{A});

L₃ is selected from the group consisting of a covalent bond, alkylene, alkenylene, O, S, C(═O), N(═OR₂₁), and N(RA);

R₂₀ is selected from the group consisting of aryl, heterocycle, and cycloalkyl;

R₂₁ is selected from the group consisting of hydrogen and alkyl;

R₂₂ is selected from the group consisting of aryl, heterocycle, and cycloalkyl;

R_{A} and R_{B} are each independently selected from the group consisting of hydrogen, alkyl, alkylcarbonyl and formyl;

provided that at least one of R₄, R₅, R₆, or R₇ is aryl, heterocycle, cycloalkyl, -L₂R₂₀ or —R₂₀L₃R₂₂.

More preferably, compounds suitable for the method of the invention have the formula (II):

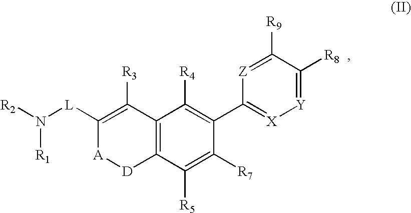

are pharmaceutically acceptable salts, esters, amides, or prodrugs thereof, wherein

R₇ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

R₈ is selected from hydrogen, alkylcarbonyl, arylcarbonyl, cyano, cycloalkylcarbonyl, heterocyclecarbonyl or (NR_{A}R_{B})carbonyl;

R₉ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

X is selected from CH, CR_{X} or N;

Y is selected from CH, CR_{Y} or N;

Z is selected from CH, CR_{Z} or N;

R_{X}, R_{Y} and R_{Z} groups are each independently selected from alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl; and

A, D, L, R_{A}, R_{B}, R₁, R₂, R₃, R₄ and R₅ are as defined in formula (I).

Preferably, compounds formula (I) and/or (II) suitable for the method of the invention are benzofuran derivatives. Specific and preferred benzofuran derivatives include, but are not limited to, 4-(2-{2-[(2R)-2-methylpyrrolidinyl]ethyl}-1-benzofuran-5-yl)benzonitrile and 4-{2-[2-(2-methyl)-1-pyrrolidinyl)ethyl]-1-benzofuran-5-yl} benzonitrile. Such compounds have demonstrated effectiveness as histamine-3 receptor ligands.

Compounds of formulae (I) and (II) can be prepared by a variety of synthetic procedures. Examples of general procedures for preparing such compounds for the method of the invention are described below in Schemes 1-5.

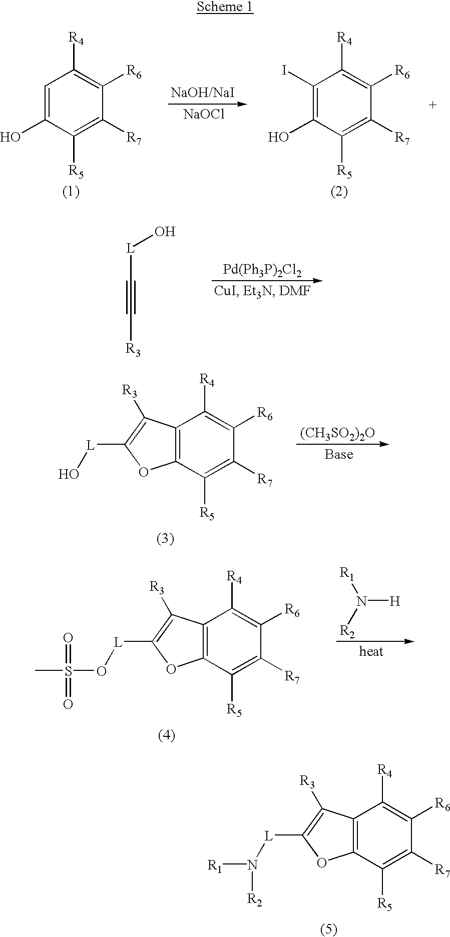

Benzofurans of general formula (5), wherein L, R₁, R₂, R₃, R₄, R₅, R₆ and R₇ are as defined in formula (I), may be prepared as described in Scheme I. Phenols of general formula (1) may be treated with sodium hypochlorite, sodium iodide and sodium hydroxide in a solvent such as methanol to provide iodides of general formula (2). Iodides of general formula (2) may be treated with substituted propargyl alcohols, dichlorobis(triphenylphosphine)palladium, copper iodide, a base such as triethylamine in a solvent such as DMF with heat to provide benzofurans of general formula (3). Alcohols of general formula (3) may be treated with methanesulfonyl chloride or methanesulfonyl anhydride, a base such as triethylamine, diisopropylethylamine or N-methylmorpholine in a solvent such as dichloromethane or THF to provide mesylates of general formula (4). Mesylates of general formula (4) may be treated with secondary or primary amines in solvents such as DMF or THF with heat to provide amines of general formula (5). Alternatively mesylates of general formula (4) may be treated with secondary or primary amine hydrochlorides in the presence of a base such as triethylamine, diisopropylethylamime or N-methylmorpholine in a solvent such as DMF or THF with heat to provide benzofurans of general formula (5).

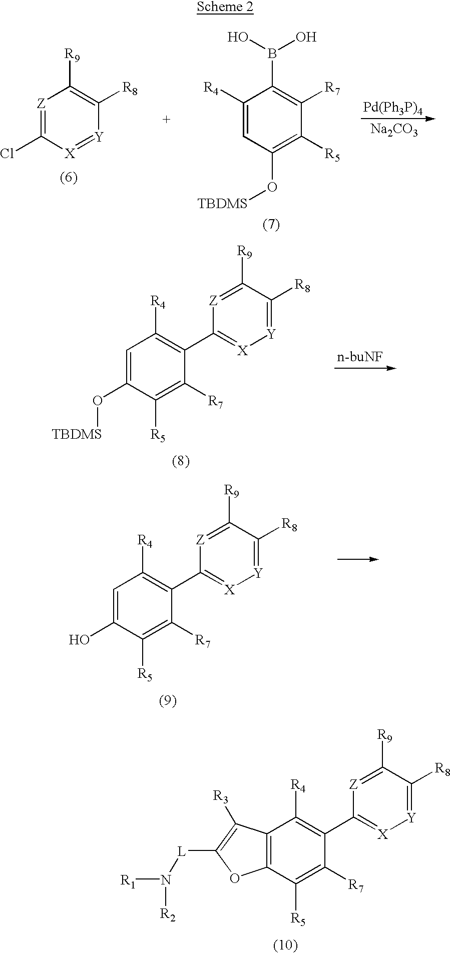

Benzofurans of general formula (10), wherein L, R₁, R₂, R₃, R₄, R₅, R₇, R₈, R₉, X, Y and Z are as defined in formula (II), may be prepared as described in Scheme 2. Chlorides of general formula (6) may be treated with boronic acids of general formula (7), tetrakis(triphenylphosphine)palladium, a base such as aqueous sodium carbonate in a solvent such as toluene with heat to provide tert-butyldimethylsilyl protected alcohols of general formula (8). Protected alcohols of general formula (8) may be treated with tetrabutylammonium fluoride in a solvent such as THF to provide alcohols of general formula (9). Alcohols of general formula (9) may be treated using conditions as described in Scheme 1 to provide benzofurans of general formula (10).

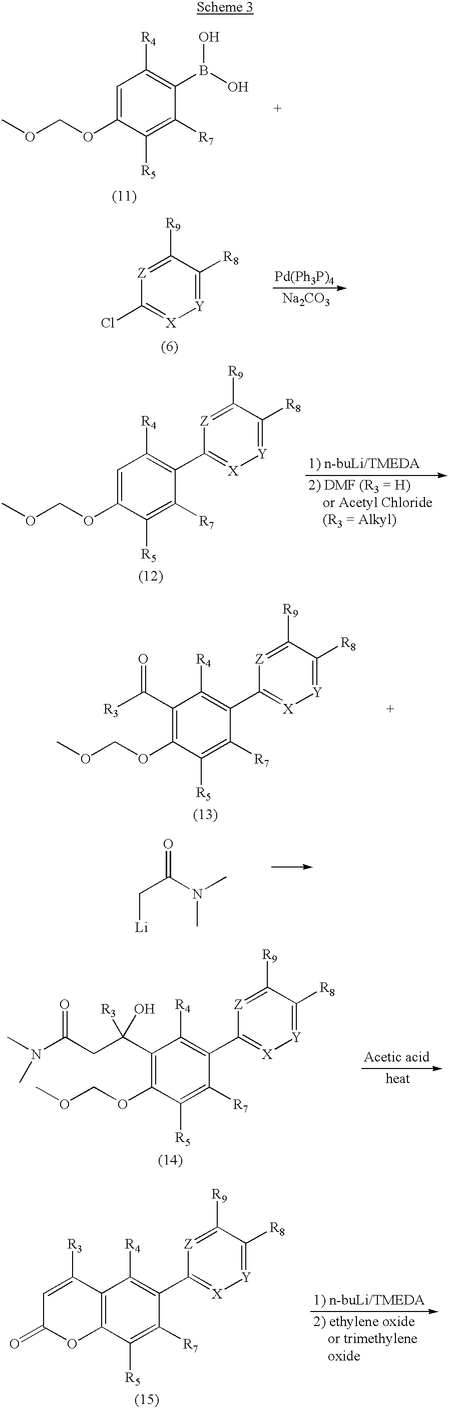

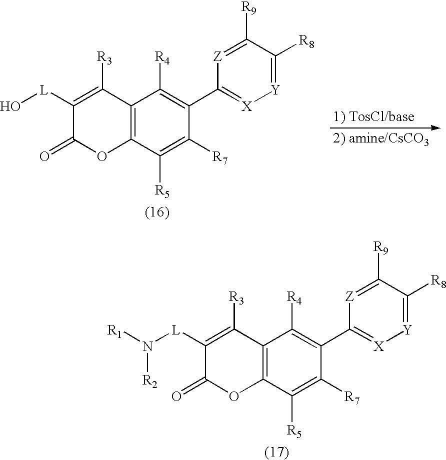

Chromenes of general formula (17), wherein L, R₁, R₂, R₃, R₄, R₅, R₇, R₈, R₉, X, Y and Z are as defined by formula (II), may be prepared as described in Scheme 3. Boronic acids of general formula (11) may be treated with chlorides of general formula (6), tetrakis(triphenylphosphine)palladium, a base such as aqueous sodium carbonate in a solvent such as toluene with heat to provide compounds of general formula (12). Compounds of general formula (12) may be treated with n-butyl lithium, N, N, N′,N′-tetramethylethylenediamine followed by DMF or acetyl chloride to provide compounds of general formula (13) which may be treated with [2-(dimethylamino)-2-oxoethyl]lithium in a solvent such as THF to provide compounds of general formula (14). Compounds of general formula (14) may be treated with acetic acid with heat to provide chromenes of general formula (15). Chromenes of general formula (15) may be treated with butyl lithium, N, N, N′,N′-tetramethylethylenediamine followed by ethylene oxide or trimethylene oxide to provide alcohols of general formula (16). Alternatively (15) may be treated with butyl lithium, N, N, N′,N′-tetramethylethylenediamine and (2-bromoethoxy) tertbutyldimethylsilane followed by tetrabutylammonium fluoride deprotection to provide alcohols of general formula (16). Alcohols of general formula (16) may be converted to the respective mesylate and further reacted with amines as described in scheme 1 to provide chromenes of general formula (17).

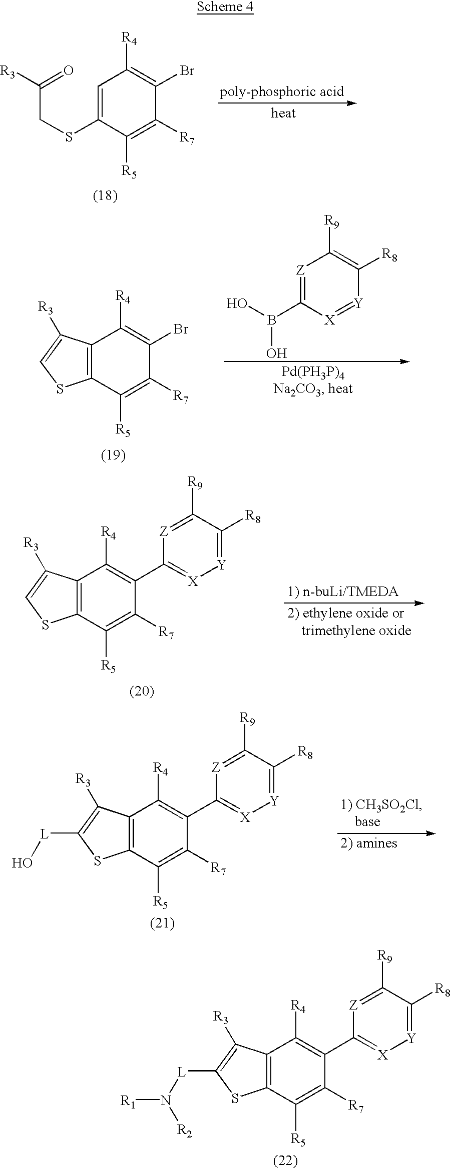

Benzothiophenes of general formula (22) wherein L, R₁, R₂, R₃, R₄, R₅, R₇, R₈, R₉, X, Y and Z are defined in formula (I), may be prepared as described in Scheme 4. Compounds of general formula (18) may be treated with poly-phosphoric acid with heat to provide benzothiophenes of general formula (19). Benzothiophenes of general formula (19) may be treated with boronic acids, tetrakis(triphenylphosphine)palladium, a base such as aqueous sodium carbonate in a solvent such as toluene with heat to provide compounds of general formula (20). Compounds of general formula (20) may be treated with n-butyl lithium, N, N, N′, N′-tetramethylethylenediamine followed by ethylene oxide to provide alcohols of general formula (21). Alcohols of general formula (21) may be converted to the mesylate and then further treated with amines as described in Scheme 1 to provide benzothiophenes of general formula (22).

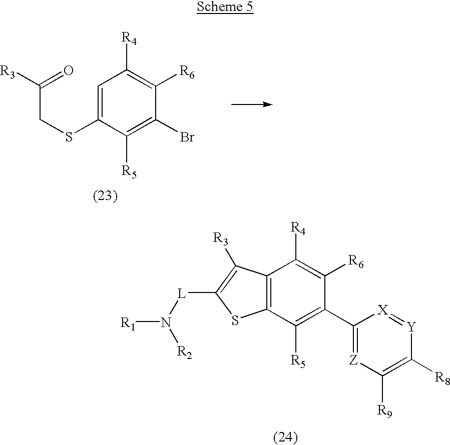

Benzothiophenes of general formula (24) wherein L, R₁, R₂, R₃, R₄, R₅, R₆, R₈, R₉ and X, Y and Z are defined in formula (I), may be prepared as described in Scheme 5. Compounds of general formula (23) may be processed as described in Scheme 4 to provide compounds of general formula (24).

One procedure suitable for preparing the preferred compound, 4-(2-{2-[(2R)-2-methylpyrrolidinyl]ethyl}-1-benzofuran-5-yl)benzonitrile, is shown in Example 1 below.

### EXAMPLE 1

### 4-(2-{2-[(2R)-2-methylpyrrolidinyl]ethyl}-1-benzofuran-5-yl)benzonitrile

### EXAMPLE 1 A

### 4′-hydroxy-3′-iodo [1,1′-biphenyl]-4-carbonitrile

To a solution of 4-hydroxy-4′-cyanobiphenyl (6.00 g, 30.8 mmol), sodium iodide (4.61 g, 30.8 mmol) and sodium hydroxide (1.23 g, 30.8 mmol) in methanol (90 mL) at 0° C. was added an aqueous solution of sodium hypochlorite (47 mL of 5.25% Clorox™, 2.29 g, 30.8 mmol) over 45 minutes. The mixture was stirred cold for 1 hour, warmed to ambient temperature and diluted with sodium thiosulfate solution (10 mL), water (80 mL) and adjusted to a pH of 7 by addition of sodium dihydrogen phosphate. The mixture was extracted with dichloromethane (2×90 mL). The combined organic extracts were dried (Na₂SO₄), filtered and concentrated under reduced pressure to give a white powder. The solid was crystallized from dichloroethane/hexane and chromatographed on silica with dichloromethane to give the titled compound (5.19 g, 53%). MS (DCI) m/z 339[M+NH₄⁺]⁺.

### EXAMPLE 1B

### 4-[2-(2-hydroxyethyl)-1-benzofuran-5-yl]benzonitrile

To a solution of Example 1 A (5.19 g, 16.2 mmol), triethylamine (5.60 mL, 40.4 mmol) and 3-butyn-1-ol (1.90 g, 27.2 mmol) in dimethylformamide (13 mL) at 20° C. was added cuprous iodide (0.46 g, 2.4 mmol) and bis-triphenylphosphine palladium dichloride (0.56 g, 0.80 mmol). The mixture was stirred at 65° C. for 12 hours then cooled to ambient temperature and diluted with dichloromethane (20 mL) and hexane (100 mL). Celite® (5 g) was added with stirring and the solids were removed by filtration. The filtrate was washed with water (600 mL). The organic layer was separated and the aqueous layer extracted with dichloromethane (3×100 mL). The combined organic solution was dried (Na₂SO₄), filtered and concentrated under reduced pressure to give a tan solid. The solid was chromatographed on silica with 3% methanol in dichloromethane to give the titled compound (4.02 g, 95%). MS (DCI) m/z 263 [M+H]⁺.

### EXAMPLE 1C

### 4-[2-(2-ethyl methanesulfonyl)-1-benzofuran-5-yl]benzonitrile

To a solution of Example 1B (0.57 g, 2.2 mmol) and triethylamine (0.9 mL, 6.5 mmol) in dichloromethane (10 mL) at 20° C. was added methane sulfonyl chloride (0.79 g, 4.5 mmol). The mixture was stirred for 30 min., diluted with dichloromethane, washed with water, dried (Na₂SO₄), filtered and concentrated under reduced pressure. The residue was chromatographed on silica with dichloromethane to give the titled compound (0.66 g, 89%). MS (DCI) m/z 359 [M+H]⁺.

### 4-(2-{2-[(2R)-2-methylpyrrolidinyl]ethyl}-1-benzofuran-5-yl)benzonitrile

A suspension of Example 1C (0.19 g, 0.55 mmol), 2-(R)-methylpyrrolidine hydrobromide (0.17 g, 1.0 mmol) and sodium carbonate (0.23 g, 2.2 mmol) in acetonitrile (0.4 mL) was heated to 50° C. with stirring for 48 hours. The reaction was cooled to ambient temperature, diluted with acetonitrile and centrifuged. The supernatant liquid was removed and the solids washed with acetonitrile. The combined liquids were concentrated under reduced pressure and the residue chromatographed by reverse phase HPLC with aqueous CF₃CO₂H/acetonitrile to give the titled compound (0.065 g, 34%). ¹H NMR (300 MHz, CD₃OD) δ 7.88 (m, 1H), 7.71 (m, 4H), 7.50 (m, 2H), 6.82 (s, 1H), 3.72-3.9 (m, 2H), 3.58 (m, 1H), 3.25-3.5 (m, 4H), 2.48 (m, 1H), 2.05-2.2 (m, 2H), 1.75 (m, 1H), 1.50 (d, J=6 Hz, 3H); MS (DCI) m/z 331 [M+H]⁺.

Compounds of formulae (I) and (II), compositions containing the same, and methods of making the compounds, or compositions thereof, are also described in copending U.S. patent application Ser. No. 09/810,648, filed Mar. 16, 2001, copending U.S. Pat. No. application Ser. No. 10,044,495, and copending U.S. patent application Ser. No. 10/081,207, filed on Feb. 22, 2002, which correspond to International Publication No. 02-074758, published Sep. 26, 2002, each of which is herein incorporated by reference in its entirety.

Aminoalkoxybiphenylcarboxamide compounds of the invention have the formula (III):

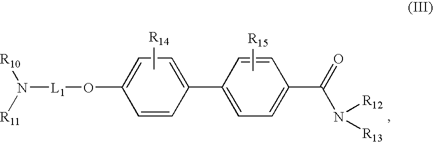

or are pharmaceutically acceptable salts, esters, amides, or prodrugs thereof, wherein

L₁ is alkylene;

R₁₀ and R₁₁ are each independently selected from the group consisting of hydrogen, alkyl, aryl, arylalkyl, cycloalkyl, cycloalkylalkyl, heterocycle and heterocyclealkyl; or

R₁₀ and R₁₁ taken together with the nitrogen atom to which they are attached, together form a heterocycle selected from the group consisting of azepanyl, azetidinyl, morpholinyl, piperazinyl, piperidinyl, pyrrolidinyl, 2,5-dihydro-1H-pyrrolyl, pyrrolyl, thiomorpholinyl and 1,1-dioxidothiomorpholinyl;

R₁₂ and R₁₃ are each independently selected from the group consisting of hydrogen, alkyl, aryl, arylalkyl, cycloalkyl, cycloalkylalkyl, heterocycle and heterocyclealkyl; or

R₁₂ and R₁₃ taken together with the nitrogen atom to which they are attached, together form a heterocycle selected from the group consisting of azepanyl, azetidinyl, morpholinyl, piperazinyl, piperidinyl, pyrrolidinyl, 2,5-dihydro-1H-pyrrolyl, pyrrolyl, thiomorpholinyl and 1,1-dioxidothiomorpholinyl; and

R₁₄ and R₁₅ are each independently selected from the group consisting of hydrogen, alkenyl, alkoxy, alkoxyalkoxy, alkoxyalkyl, alkoxycarbonyl, alkyl, alkylcarbonyl, alkylcarbonyloxy, alkylsulfinyl, alkylsulfonyl, alkylthio, alkynyl, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;

provided that when R₁₀ and R₁₁ together form pyrrolidinyl and wherein said pyrrolidinyl is substituted with one substituent then said substituent is other than alkoxy, hydroxy or —NR_{A}R_{B}.

Compounds of formula (III) are further described in U.S. Pat. No. 6,316,475, issued Nov. 13, 2001, which is herein incorporated by reference in its entirety. Methods for preparing the compounds also are described in U.S. Pat. No. 6,316,475. Aminoalkoxybiphenylcarboxamide compounds can be prepared by a variety of synthetic routes including, for example, the procedure shown in Scheme 6.

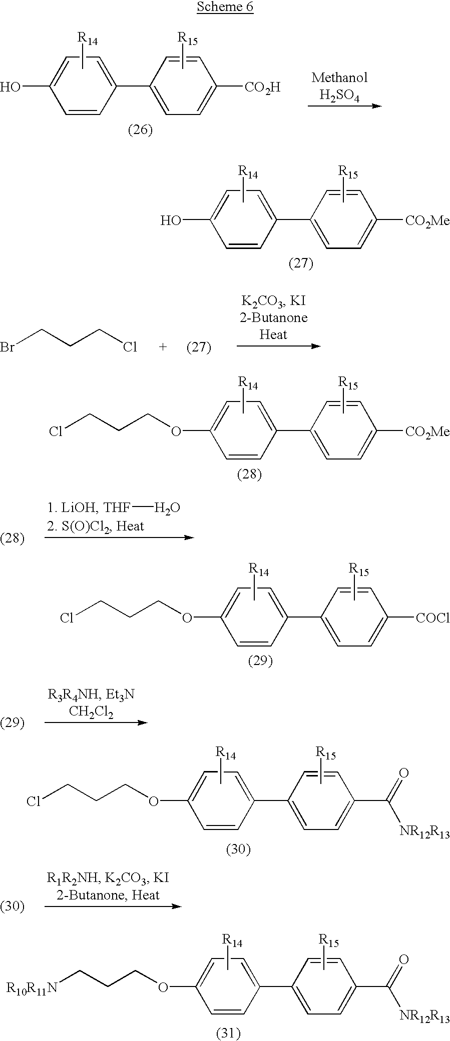

Compounds of general formula (31), wherein R₁₀, R₁₁, R₁₂, R₁₃, R₁₄ and R₁₅ are as defined in formula (III), may be prepared as described in Scheme 6. Compounds of general formula (26), which may be purchased or prepared using standard chemistry known to those in the art, may be treated with sulfuric acid in methanol to provide esters of general formula (27). Esters of general formula (27) may be treated with 1-bromo-3-chloropropane (or 1-bromo-2-chloroethane to provide the ethoxy analogues or still another appropriate bromo-chloroalkane to provide analogues as defined in formula (III)), potassium carbonate, and potassium iodide in 2-butanone at reflux for about 24 hours to provide chlorides of general formula (28). Chlorides of general formula (28) may be treated with lithium hydroxide in THF:H₂O (3:1) to provide the crude acids. The crude acids may be treated with thionyl chloride (used as solvent) and heat (about 90° C.) for about 4 hours in to provide acid chlorides of general formula (29). Acid chlorides of general formula (29) may be treated with a base such as triethylamine and amines of general formula R₁₂R₁₃NH in a solvent such as methylene chloride to provide amides of general formula (30). Amides of general formula (30) may be treated with a base such as potassium carbonate, potassium iodide and a base of general formula R₁₀R₁₁NH in a solvent such as 2-butanone with heat to provide compounds of general formula (31).

Aminoetherbiphenyl compounds also are suitable for the invention. Such compounds have the formula (IV):

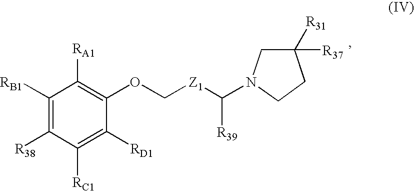

or a pharmaceutically acceptable salt thereof, wherein

Z₁ is selected from a covalent bond or CH₂;

R₃, is selected from OR₃₂, NR₃₃R₃₄ or

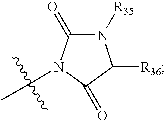

R₃₂ is selected from hydrogen, alkoxycarbonyl, alkyl, alkylcarbonyl, aminocarbonyl, sulfono or phosphono;

R₃₃ and R₃₄ are independently selected from hydrogen, alkenyl, alkenylcarbonyl, alkenyloxycarbonyl, alkenylsulfonyl, alkoxycarbonyl, alkyl, alkylcarbonyl, alkylsulfonyl, alkynyl, alkynylcarbonyl, alkynyloxycarbonyl, alkynylsulfonyl, aminocarbonyl, aminosulfonyl, arylalkyl, arylalkenylcarbonyl, arylalkenylsulfonyl, arylalkylcarbonyl, arylalkylsulfonyl, arylarylcarbonyl, arylarylsulfonyl, arylcarbonyl, arylheterocylecarbonyl, arylheterocylesulfonyl, aryloxyarylcarbonyl, aryloxyarylsulfonyl, arylsulfonyl, cycloalkyl, cycloalkylalkyl, cycloalkylalkylcarbonyl, cycloalkylalkylsulfonyl, cycloalkylcarbonyl, cycloalkylsulfonyl, formyl, heterocycle, heterocyclealkyl, heterocyclealkylcarbonyl, heterocyclealkylsulfonyl, heterocyclearylcarbonyl, heterocyclearylsulfonyl, heterocyclecarbonyl, heterocycleheterocyclecarbonyl, heterocycleheterocyclesulfonyl, heterocycleoxyalkylcarbonyl, heterocycleoxyarylcarbonyl, heterocycleoxyarylsulfonyl, heterocyclesulfonyl, or heterocyclethioalkylcarbonyl;

R₃₅ and R₃₆ are independently selected from hydrogen or alkyl;

R₃₇ is selected from hydrogen or alkyl; or

R₃₁ and R₃₇ together form (═O);

R₃₈ is selected from alkylcarbonyl, aryl, arylcarbonyl, arylcarbonylaryl, arylcarbonylheterocycle, cycloalkylcarbonyl, cycloalkylcarbonylaryl, cycloalkylcarbonylheterocycle, heterocycle, heterocyclecarbonyl, heterocyclecarbonylaryl or heterocyclecarbonylheterocycle;

R₃₉ is selected from the hydrogen or lower alkyl; and

R_{A1}, R_{B1}, R_{C1} and R_{D1} are independently selected from hydrogen, alkenyl, alkoxy, alkoxyalkoxy, alkoxyalkyl, alkoxycarbonyl, alkyl, alkylcarbonyl, alkylcarbonyloxy, alkylsulfinyl, alkylsulfonyl, alkylthio, alkynyl, amino, aminoalkyl, aminocarbonyl, carboxy, carboxyalkyl, cyano, cyanoalkyl, formyl, halogen, haloalkoxy, haloalkyl, hydroxy, hydroxyalkyl, mercapto or nitro.

A preferred compound of formula (IV) suitable for the method of the invention includes, but is not limited to, 4′-{3-[(3R)-3-(dimethylamino)pyrrolidinyl]propoxy}[1,1′-biphenyl]-4-carbonitrile which demonstrated effectiveness as a histamine-3 receptor ligand.

Compounds of formula (IV) and suitable methods for preparing such compounds are further described in International Publication WO 02/06223, published Jan. 24, 2002, and U.S. Publication 2002-0137931-A1, published Sep. 26, 2002, each of which is herein incorporated by reference in its entirety. Aminoetherbiphenyl compounds can be prepared by a variety of synthetic routes including, for example, the procedure shown in Scheme 7.

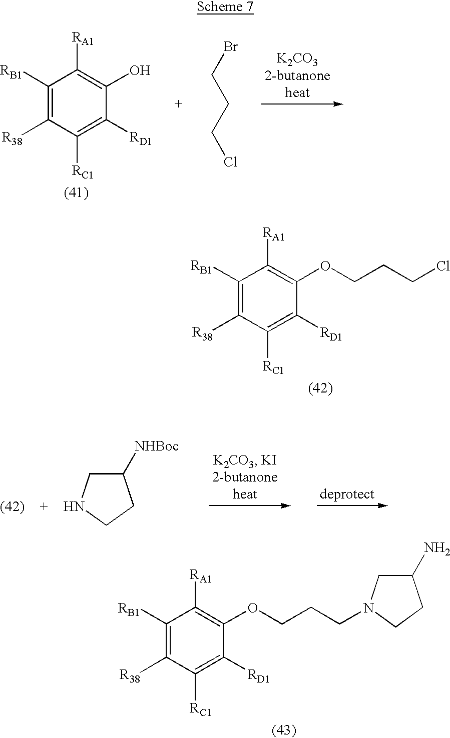

Aminoetherbiphenyls of general formula (43), wherein R₃₈, R_{A1}, R_{B1}, R_{C1} and R_{D1} are as defined in formula (IV) may be prepared as described in Scheme 7. Phenols of general formula (41), obtained commercially or prepared using standard methodology known to those of skill in the art, may be treated with 1-bromo-3-chloropropane (or 1-bromo-2-chloroethane to provide the ethyl analogues) and a base such as potassium carbonate in a solvent such as 2-butanone with heat to provide chlorides of general formula (42). Chlorides of general formula (42) may be treated with tert-butyl pyrrolidinylcarbamate (or tert-butyl (3R)-pyrrolidinylcarbamate or tert-butyl (3S)-pyrrolidinylcarbamate), potassium iodide, a base such as potassium carbonate in a solvent such as 2-butanone with heat to provide N-boc aminopyrrolidines which may be deprotected with acid such as 4N HCl in 1,4-dioxane or trifluoroacetic acid in CH₂Cl₂ to provide aminoetherbiphenyls of general formula (43). Typically, substituents for R₃₈ on compounds of general formula (42) can be prepared by a coupling reaction in the presence of a transition metal catalyst such as tetrakis(triphenylphosphine) palladium and a base such as potassium carbonate or cesium carbonate under standard Suzuki, Stille or Heck coupling conditions well known to those of skill in the art to provide substiuents such as, but not limited to, 4-cyanophenyl.

One procedure suitable for preparing the preferred aminoetherbiphenyl compound, 4′-{3-[(3R)-3-(dimethylamino)pyrrolidinyl]propoxy} [1,1′-biphenyl]-4-carbonitrile is shown in Example 2.

### EXAMPLE 2

### 4′-{3-[(3R)-3-(dimethylamino)pyrrolidinyl]propoxy}[1,1′-biphenyl]-4-carbonitrile

4′-(3-Chloropropoxy)-1,1′-biphenyl-4-carbonitrile (200 mg, 0.74 mmol), N,N-dimethyl-N-[(3R)-pyrrolidinyl]amine (85 mg, 0.74 mmol), 250 mg of potassium carbonate and 300 mg of potassium iodide in 20 mL of 2-butanone were heated at 110° C. for 72 hours. The mixture was evaporated under reduced pressure and the residue was purified by chromatography (CHCl₃:MeOH:NH₄OH, 9:1:0.1) to provide the title compound. MS (ESI+) m/z 350 (M+H)⁺; ¹³C NMR(500 MHz, CD₃OD) 29.3, 29.6, 43.9, 54.2, 54.3, 59.6, 66.5, 67.3, 111.0, 116.2, 119.9, 128.2, 129.4, 132.7, 133.7, 146.7, 161.2; ¹HNMR (500 MHz, CD₃OD) 1.74 (m, 1H), 2.0 (m, 2H), 2.02 (m, 1H) 2.23 (s, 6H), 2.32 (m, 1H), 2.51 (m, 1H), 2.62 (m, 1H), 2.71 (m, 1H), 2.84 (m, 2H), 2.97 (m, 1H), 4.08 (t, J=7 Hz, 2H), 7.02 (d, J=11 Hz, 2H), 7.61 (d, J=1 Hz, 2H), 7.74 (s, 4H).

### Definition of Terms

As used for the present invention, the following terms have the meanings ascribed.

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl and 3-decenyl.

The term “alkenylcarbonyl,” as used herein, refers to an alkenyl group, as defined herein, appended to the parent molecular moiety through a carbonyl group, as defined herein. Representative examples of alkenylcarbonyl include, but are not limited to, 3-butenoyl, 3-pentenoyl, and 4-pentenoyl.

The term “alkenyloxy,” as used herein, refers to an alkenyl group, as defined herein, appended to the parent molecular moiety through an oxy group, as defined herein. Representative examples of alkenyloxy include, but are not limited to, allyloxy, 2-butenyloxy, and 3-butenyloxy.

The term “alkenyloxycarbonyl,” as used herein, refers to an alkenyloxy group, as defined herein, appended to the parent molecular moiety through a carbonyl group, as defined herein. Representative examples of alkenyloxycarbonyl include, but are not limited to, allyloxycarbonyl, 2-butenyloxycarbonyl, and 3-butenyloxycarbonyl.

The term “alkenylsulfonyl,” as used herein, refers to an alkenyl group, as defined herein, appended to the parent molecular moiety through a sulfonyl group, as defined herein. Representative examples of alkenylsulfonyl include, but are not limited to, allylsulfonyl, 2-butenylsulfonyl, and 3-butenylsulfonyl.

The term “alkenylene” means a divalent group derived from a straight or branched chain hydrocarbon of from 2 to 10 carbon atoms containing at least one double bond. Representative examples of alkenylene include, but are not limited to, —CH═CH—, —C(═CH₂)—, —CH═CH₂CH₂, —CH₂CH₂C(═CH₂)CH₂, —CH₂CH₂C(═CHCH₃)CH₂—, and —CH═C(CH₃)CH₂—.

The term “alkoxyalkoxy,” as used herein, refers to an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy and methoxymethoxy.

The term “alkoxy,” as used herein, refers to an alkyl group, as defined herein, appended to the parent molecular moiety through an oxy moiety, as defined herein. Representative examples of alkoxy include, but are not limited to, methoxy, ethoxy, propoxy, 2-propoxy, butoxy, tert-butoxy, pentyloxy and hexyloxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl and tert-butoxycarbonyl.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl and 1-oxopentyl.

The term “alkylcarbonyloxy,” as used herein, refers to an alkylcarbonyl group, as defined herein, appended to the parent molecular moiety through an oxy moiety, as defined herein. Representative examples of alkylcarbonyloxy include, but are not limited to, acetyloxy, ethylcarbonyloxy and tert-butylcarbonyloxy.

The term “alkylene” means a divalent group derived from a straight or branched chain hydrocarbon of from 1 to 10 carbon atoms. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkylsulfinyl,” as used herein, refers to an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfonyl,” as used herein, refers to an alkyl group, as defined herein, appended to the parent molecular moiety through a sulfonyl group, as defined herein. Representative examples of alkylsulfonyl include, but are not limited to, ethylsulfonyl, isopropylsulfonyl and methylsulfonyl.

The term “alkylthio,” as used herein, refers to an alkyl group, as defined herein, appended to the parent molecular moiety through a sufur atom, as defined herein. Representative examples of alkylthio include, but are not limited to, methylsulfanyl, ethylsulfanyl, tert-butylsulfanyl and hexylsulfanyl.

The term “alkynyl” as used herein refers to straight or branched chain hydrocarbon group containing from 2 to 10 carbon atoms and containing at least on carbon-carbon triple bond. Representative examples of alkynyl include, but are not limited to, acetylenyl, 1-propynyl, 2-propynyl, 3-butynyl, 2-pentynyl and 1-butynyl.

The term “alkynylcarbonyl,” as used herein, refers to an alkynyl group, as defined herein, appended to the parent molecular moiety through a carbonyl group, as defined herein. Representative examples of alkynylcarbonyl include, but are not limited to, 3-butynoyl, 3-pentynoyl, and 4-pentynoyl.

The term “alkynyloxy,” as used herein, refers to an alkynyl group, as defined herein, appended to the parent molecular moiety through an oxy group, as defined herein. Representative examples of alkynyloxy include, but are not limited to, 2-butynyloxy, and 3-butynyloxy.

The term “alkynyloxycarbonyl,” as used herein, refers to an alkynyloxy group, as defined herein, appended to the parent molecular moiety through a carbonyl group, as defined herein. Representative examples of alkynyloxycarbonyl include, but are not limited to, 2-butynyloxycarbonyl, and 3-butynyloxycarbonyl.

The term “alkynylsulfonyl,” as used herein, refers to an alkynyl group, as defined herein, appended to the parent molecular moiety through a sulfonyl group, as defined herein. Representative examples of alkynylsulfonyl include, but are not limited to, 2-butynylsulfonyl, and 3-butynylsulfonyl.

The term “amino,” as used herein, refers to a —NR₄₀R₄₁ group wherein R₄₀ and R₄₁ are independently selected from hydrogen, alkyl, alkylcarbonyl, and benzyl. Representative examples of amino include but are not limited to acetylamino, amino, benzylamino, dimethylamino, and methylamino.

The term “aminoalkyl,” as used herein, refers to an amino group, as defined herein, appended to the parent molecular moiety through an alkyl group, as defined herein. Representative examples of aminoalkyl include, but are not limited, (amino)methyl, (dimethylamino)methyl, 2-(benzylamino)ethyl, and (ethylamino)methyl.

The term “aminocarbonyl,” as used herein, refers to an amino group, as defined herein, appended to the parent molecular moiety through a carbonyl group, as defined herein. Representative examples of aminocarbonyl include, but are not limited, aminocarbonyl, dimethylaminocarbonyl, benzylaminocarbonyl, and ethylaminocarbonyl.

The term “aminosulfonyl,” as used herein, refers to an amino group, as defined herein, appended to the parent molecular moiety through a sulfonyl group, as defined herein. Representative examples of aminosulfonyl include, but are not limited, aminosulfonyl, dimethylaminosulfonyl, benzylaminosulfonyl, and ethylaminosulfonyl.

The term “aryl,” as used herein, refers to a monocyclic-ring system, or a bicyclic- or a tricyclic-fused ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of this invention can be substituted with 1, 2, 3, 4 or 5 substituents independently selected from alkenyl, alkoxy, alkoxyalkoxy, alkoxyalkyl, alkoxycarbonyl, alkyl, alkylcarbonyl, alkylcarbonyloxy, alkylsulfinyl, alkylsulfonyl, alkylthio, alkynyl, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl.

The term “arylalkenyl,” as used herein, refers to an aryl group, as defined herein, appended to the parent molecular moiety through an alkenyl group, as defined herein. Representative examples of arylalkenyl include, but are not limited to, 3-phenyl-1-propenyl, and 2-(2-naphthyl)ethenyl.

The term “arylalkenylcarbonyl,” as used herein, refers to an arylalkenyl group, as defined herein, appended to the parent molecular moiety through a carbonyl group, as defined herein. Representative examples of arylalkenylcarbonyl include, but are not limited to, 4-phenyl-3-butenoyl, and 3-phenyl-2-propenoyl.

The term “arylalkenylsulfonyl,” as used herein, refers to an arylalkenyl group, as defined herein, appended to the parent molecular moiety through a sulfonyl group, as defined herein. Representative examples of arylalkenylsulfonyl include, but are not limited to, 2-phenylethenylsulfonyl, and 4-phenyl-3-butenylsulfonyl.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl, phenylacetyl, 3-phenylpropionyl and 2-naphthylacetyl.

The term “arylalkylcarbonyl,” as used herein, refers to an arylalkyl group, as defined herein, appended to the parent molecular moiety through a carbonyl group, as defined herein. Representative examples of arylalkylcarbonyl include, but are not limited to, phenylacetyl, 4-phenylbutanoyl, and 3-phenylpropanoyl.

The term “arylalkylsulfonyl,” as used herein, refers to an arylalkyl group, as defined herein, appended to the parent molecular moiety through a sulfonyl group, as defined herein. Representative examples of arylalkylsulfonyl include, but are not limited to, (2-phenylethyl)sulfonyl, and (3-phenylpropyl)sulfonyl.

The term “arylaryl,” as used herein, refers to an aryl group, as defined herein, appended to the parent molecular moiety through another aryl group, as defined herein. Representative examples of arylaryl include, but are not limited to, (1,1′-biphenyl), and (2′-chloro(1,1′-biphenyl)-3-yl).

The term “arylarylcarbonyl,” as used herein, refers to an arylaryl group, as defined herein, appended to the parent molecular moiety through a carbonyl group, as defined herein. Representative examples of arylarylcarbonyl include, but are not limited to, (1,1′-biphenyl)carbonyl, and (2′-chloro(1,1′-biphenyl)-3-yl)carbonyl.

The term “arylarylsulfonyl,” as used herein, refers to an arylaryl group, as defined herein, appended to the parent molecular moiety through a sulfonyl group, as defined herein. Representative examples of arylarylsulfonyl include, but are not limited to, (1,1′-biphenyl)sulfonyl, and (2′-chloro(1,1′-biphenyl)-3-yl)sulfonyl.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl, 4-cyanobenzoyl, and naphthoyl.

The term “arylcarbonylaryl,” as used herein, refers to an arylcarbonyl group, as defined herein, appended to the parent molecular moiety through an aryl group, as defined herein. Representative examples of arylcarbonylaryl include, but are not limited to, 4-(benzoyl)phenyl and 4-(benzoyl)naphthyl.

The term “arylcarbonylheterocycle,” as used herein, refers to an arylcarbonyl group, as defined herein, appended to the parent molecular moiety through a heterocycle group, as defined herein. Representative examples of arylcarbonylheterocycle include, but are not limited to, 4-benzoyl-1-piperazinyl and 1-benzoyl-4-piperidinyl.

The term “arylheterocycle,” as used herein, refers to an aryl group, as defined herein, appended to the parent molecular moiety through a heterocycle group, as defined herein. Representative examples of arylheterocycle include, but are not limited to, 5-phenylpyridin-2-yl and 5-(3-chlororphenyl)pyridin-2-yl.

The term “arylheterocyclecarbonyl,” as used herein, refers to an arylheterocycle group, as defined herein, appended to the parent molecular moiety through a carbonyl group, as defined herein. Representative examples of arylheterocyclecarbonyl include, but are not limited to, 5-phenylpyridin-2-ylcarbonyl and 5-(3-chlororphenyl)pyridin-2-ylcarbonyl.

The term “arylheterocyclesulfonyl,” as used herein, refers to an arylheterocycle group, as defined herein, appended to the parent molecular moiety through a sulfonyl group, as defined herein. Representative examples of arylheterocyclesulfonyl include, but are not limited to, 5-phenylpyridin-2-ylsulfonyl and 5-(3-chlororphenyl)pyridin-2-ylsulfonyl.

The term “aryloxy,” as used herein, refers to an aryl group, as defined herein, appended to the parent molecular moiety through an oxy moiety, as defined herein. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “aryloxyaryl,” as used herein, refers to an aryloxy group, as defined herein, appended to the parent molecular moiety through an aryl group, as defined herein. Representative examples of aryloxyaryl include, but are not limited to, 3-(3-methylphenoxy)phenyl, and 3-(3-bromophenoxy)phenyl.

The term “aryloxyarylcarbonyl,” as used herein, refers to an aryloxyaryl group, as defined herein, appended to the parent molecular moiety through a carbonyl group, as defined herein. Representative examples of aryloxyarylcarbonyl include, but are not limited to, 3-(3-methylphenoxy)benzoyl, and 3-(3-bromophenoxy)benzoyl.

The term “aryloxyarylsulfonyl,” as used herein, refers to an aryloxyaryl group, as defined herein, appended to the parent molecular moiety through a sulfonyl group, as defined herein. Representative examples of aryloxyarylsulfonyl include, but are not limited to, 3-(3-methylphenoxy)phenylsulfonyl, and 3-(3-bromophenoxy)phenylsulfonyl.

The term “arylsulfonyl,” as used herein, refers to an aryl group, as defined herein, appended to the parent molecular moiety through a sulfonyl group, as defined herein. Representative examples of arylsulfonyl include, but are not limited to, phenylsulfonyl, (4-acetylaminophenyl)sulfonyl, (4-chlorophenyl)sulfonyl, (4-cyanophenyl)sulfonyl, (4-methoxyphenyl)sulfonyl, (4-methylphenyl)sulfonyl, and (4-(tert-butyl)phenyl)sulfonyl.

The term “arylthio,” as used herein, refers to an aryl group, as defined herein, appended to the parent molecular moiety through a thio moiety, as defined herein. Representative examples of arylthio include, but are not limited to, phenylsulfanyl, naphth-2-ylsulfanyl, and 5-phenylhexylsulfanyl.

The term “carbonyl,” as used herein, refers to a —C(O)— group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano,” as used herein, refers to a —CN group.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl and 3-cyanopropyl.

The term “cycloalkyl,” as used herein, refers to a saturated cyclic hydrocarbon group containing from 3 to 8 carbons. Examples of cycloalkyl include cyclopropyl, cyclobutyl, cyclopentyl, cyclohexyl, cycloheptyl and cyclooctyl.

The term “cycloalkylalkyl,” as used herein, refers to cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl and 4-cycloheptylbutyl.

The term “cycloalkylalkylcarbonyl,” as used herein, refers to a cycloalkylalkyl group, as defined herein, appended to the parent molecular moiety through a carbonyl group, as defined herein. Representative examples of cycloalkylalkylcarbonyl include, but are not limited to, cyclopropylmethylcarbonyl, 2-cyclobutylethylcarbonyl, cyclopentylmethylcarbonyl, cyclohexylmethylcarbonyl, and 4-cycloheptylbutylcarbonyl.

The term “cycloalkylalkylsulfonyl,” as used herein, refers to a cycloalkylalkyl group, as defined herein, appended to the parent molecular moiety through a sulfonyl group, as defined herein. Representative examples of cycloalkylalkylsulfonyl include, but are not limited to, cyclopropylmethylsulfonyl, 2-cyclobutylethylsulfonyl, cyclopentylmethylsulfonyl, cyclohexylmethylsulfonyl, and 4-cycloheptylbutylsulfonyl.

The term “cycloalkylcarbonyl,” as used herein, refers to a cycloalkyl group, as defined herein, appended to the parent molecular moiety through a carbonyl group, as defined herein. Representative examples of cycloalkylcarbonyl include, but are not limited to cyclopropylcarbonyl, cyclobutylcarbonyl, cyclopentylcarbonyl and cyclohexylcarbonyl.

The term “cycloalkylcarbonylaryl,” as used herein, refers to a cycloalkylcarbonyl group, as defined herein, appended to the parent molecular moiety through an aryl group, as defined herein. Representative examples of cycloalkylcarbonylaryl include, but are not limited to, 4-(cyclopropylcarbonyl)phenyl, 4-(cyclopentylcarbonyl)phenyl, and 4-(cyclohexylcarbonyl)phenyl.

The term “cycloalkylcarbonylheterocycle,” as used herein, refers to a cycloalkylcarbonyl group, as defined herein, appended to the parent molecular moiety through a heterocycle group, as defined herein. Representative examples of cycloalkylcarbonylheterocycle include, but are not limited to, 4-(cyclopropylcarbonyl)-1-piperazinyl, 4-(cyclopentylcarbonyl)-1-piperazinyl, and 4-(cyclohexylcarbonyl)-1-piperazinyl.

The term “cycloalkylsulfonyl,” as used herein, refers to cycloalkyl group, as defined herein, appended to the parent molecular moiety through a sulfonyl group, as defined herein. Representative examples of cycloalkylsulfonyl include, but are not limited to, cyclopropylsulfonyl, cyclopentylsulfonyl, and cyclohexylsulfonyl.

The term “fluoroalkylene” means an alkylene, as defined herein, containing 1 or fluorine atoms. Representative examples of fluoroalkylene include, but are not limited to, —CH₂CH(F)—, —CH₂C(F)₂—, —CH₂C(F)₂CH₂—, and —CH₂CH₂C(F)₂—.

The term “formyl,” as used herein, refers to a —C(O)H group. The term “halo” or “halogen,” as used herein, refers to —Cl, —Br, —I or —F.

The term “haloalkoxy,” as used herein, refers to at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl,” as used herein, refers to at least one halogen, as defined herein, appended to the parent molecular moiety through an alkyl group, as defined herein. Representative examples of haloalkyl include, but are not limited to, chloromethyl,-2-fluoroethyl, trifluoromethyl, pentafluoroethyl, and 2-chloro-3-fluoropentyl.

The term “heterocycle” or “heterocyclic,” as used herein, refers to a monocyclic or bicyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered rings have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridyl, pyrimidinyl, pyridazinyl, 2,5-dihydro-1H-pyrrolyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic heterocyclic ring systems fused to an aryl group as defined herein, a cycloalkyl group as defined herein, or another monocyclic heterocyclic ring system. Representative examples of bicyclic ring systems include but are not limited to, benzimidazolyl, benzothiazolyl; benzothienyl, benzoxazolyl, benzofuranyl, benzopyranyl, benzothiopyranyl, benzodioxinyl, 1,3-benzodioxolyl, cinnolinyl, indazolyl, indolyl, indolinyl, indolizinyl, naphthyridinyl, isobenzofuranyl, isobenzothienyl, isoindolyl, isoindolinyl, isoquinolinyl, phthalazinyl, pyranopyridyl, quinolinyl, quinolizinyl, quinoxalinyl, quinazolinyl, tetrahydroisoquinolinyl, tetrahydroquinolinyl, and thiopyranopyridyl.

The heterocycles of this invention can be substituted with 1, 2, or 3 substituents independently selected from alkenyl, alkoxy, alkoxyalkyl, alkoxycarbonyl, alkyl, alkylcarbonyl, alkylcarbonyloxy, alkylsulfinyl, alkylsulfonyl, alkylthio, arylalkyl, carboxy, carboxyalkyl, cyano, cyanoalkyl, formyl, halogen, haloalkoxy, haloalkyl, hydroxy, hydroxyalkyl, mercapto, nitro, oxo, —NR_{A}R_{B}, (NR_{A}R_{B})alkyl, (NR_{A}R_{B})carbonyl and (NR_{A}R_{B})sulfonyl.

The term “heterocyclealkyl,” as used herein, refers to a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl.

The term “heterocyclecarbonyl,” as used herein, refers to a heterocycle, as defined herein, appended to the parent molecular moiety through a carbonyl group, as defined herein. Representative examples of heterocyclecarbonyl include, but are not limited to, 1H-imidazol-1-ylcarbonyl, 4-morpholinylcarbonyl, 1-piperidinylcarbonyl and cyclopentylaminocarbonyl.

The term “heterocyclealkylsulfonyl,” as used herein, refers to a heterocyclealkyl, as defined herein, appended to the parent molecular moiety through a sulfonyl group, as defined herein. Representative examples of heterocyclealkylsulfonyl include, but are not limited to, (pyridin-3-ylmethyl)sulfonyl and (2-(pyrimidin-2-yl)propyl)sulfonyl.

The term “heterocyclearyl,” as used herein, refers to a heterocycle, as defined herein, appended to the parent molecular moiety through an aryl group, as defined herein. Representative examples of heterocyclearyl include, but are not limited to, 4-(pyridin-3-yl)phenyl and 4-(pyrimidin-2-yl)phenyl.

The term “heterocyclearylcarbonyl,” as used herein, refers to a heterocyclearyl group, as defined herein, appended to the parent molecular moiety through a carbonyl group, as defined herein. Representative examples of heterocyclearylcarbonyl include, but are not limited to, 4-(pyridin-3-yl)benzoyl and 4-(pyrimidin-2-yl)benzoyl.

The term “heterocyclearylsulfonyl,” as used herein, refers to a heterocyclearyl group, as defined herein, appended to the parent molecular moiety through a sulfonyl group, as defined herein. Representative examples of heterocyclearylsulfonyl include, but are not limited to, (4-(pyridin-3-yl)phenyl)sulfonyl and (4-(pyrimidin-2-yl)phenyl)sulfonyl.

The term “heterocyclecarbonylaryl,” as used herein, refers to a heterocyclecarbonyl, as defined herein, appended to the parent molecular moiety through an aryl group, as defined herein. Representative examples of heterocyclecarbonylaryl include, but are not limited to, 4-(2-furoyl)phenyl, 4-(1-pyrrolidinylcarbonyl)phenyl, 4-(1-piperidinylcarbonyl)phenyl, 4-(4-morpholinylcarbonyl)phenyl, 4-(1-azetidinylcarbonyl)phenyl, 4-(1-piperazinylcarbonyl)phenyl and 4-(3-pyridinylcarbonyl)phenyl.

The term “heterocyclecarbonylheterocycle,” as used herein, refers to a heterocyclecarbonyl, as defined herein, appended to the parent molecular moiety through a heterocycle group, as defined herein. Representative examples of heterocyclecarbonylheterocycle include, but are not limited to, 4-(2-furoyl)-1-piperazinyl, 4-(1-pyrrolidinylcarbonyl)-1-piperazinyl, 4-(1-piperidinylcarbonyl)-1-piperazinyl, 4-(4-morpholinylcarbonyl)-1-piperazinyl, 4-(1-azetidinylcarbonyl)-1-piperazinyl, 4-(1-piperazinylcarbonyl)-1-piperazinyl and 4-(3-pyridinylcarbonyl)-1-piperazinyl.

The term “heterocycleheterocycle,” as used herein, refers to a heterocycle group, as defined herein, appended to the parent molecular moiety through another heterocycle group, as defined herein. Representative examples of heterocycleheterocycle include, but are not limited to, 2-(pyridin-3-yl)thiazo-4-yl and 2-(pyrimidin-2-yl)thiazo-4-yl.

The term “heterocycleheterocyclecarbonyl,” as used herein, refers to a heterocycleheterocycle group, as defined herein, appended to the parent molecular moiety through a carbonyl group, as defined herein. Representative examples of heterocycleheterocyclecarbonyl include, but are not limited to, (2-(pyridin-3-yl)thiazo-4-yl)carbonyl and (2-(pyrimidin-2-yl)thiazo-4-yl)carbonyl.

The term “heterocycleheterocyclesulfonyl,” as used herein, refers to a heterocycleheterocycle group, as defined herein, appended to the parent molecular moiety through a sulfonyl group, as defined herein. Representative examples of heterocycleheterocyclesulfonyl include, but are not limited to, (2-(pyridin-3-yl)thiazo-4-yl)sulfonyl and (2-(pyrimidin-2-yl)thiazo-4-yl)sulfonyl.

The term “heterocycleoxy,” as used herein, refers to a heterocycle group, as defined herein, appended to the parent molecular moiety through an oxy moiety, as defined herein. Representative examples of heterocycleoxy include, but are not limited to, pyrid-3-yloxy and quinolin-3-yloxy.

The term “heterocycleoxyalkyl,” as used herein, refers to a heterocycleoxy group, as defined herein, appended to the parent molecular moiety through an alkyl group, as defined herein. Representative examples of heterocycleoxyalkyl include, but are not limited to, pyrid-3-yloxymethyl and 2-quinolin-3-yloxyethyl.

The term “heterocycleoxyalkylcarbonyl,” as used herein, refers to a heterocycleoxyalkyl group, as defined herein, appended to the parent molecular moiety through a carbonyl group, as defined herein. Representative examples of heterocycleoxyalkylcarbonyl include, but are not limited to, (pyridin-3-yloxymethyl)carbonyl and (2-(quinolin-3-yloxy)ethyl)carbonyl.

The term “heterocycleoxyaryl,” as used herein, refers to a heterocycleoxy group, as defined herein, appended to the parent molecular moiety through an aryl group, as defined herein. Representative examples of heterocycleoxyaryl include, but are not limited to, 4-(pyridin-3-yloxy)phenyl and 4-(quinolin-3-yloxy)phenyl.

The term “heterocycleoxyarylcarbonyl,” as used herein, refers to a heterocycleoxyaryl group, as defined herein, appended to the parent molecular moiety through a carbonyl group, as defined herein. Representative examples of heterocycleoxyarylcarbonyl include, but are not limited to, 4-(pyridin-3-yloxy)benzoyl and 4-(quinolin-3-yloxy)benzoyl.

The term “heterocycleoxyarylsulfonyl,” as used herein, refers to a heterocycleoxyaryl group, as defined herein, appended to the parent molecular moiety through a sulfonyl group, as defined herein. Representative examples of heterocycleoxyarylsulfonyl include, but are not limited to, (4-(pyridin-3-yloxy)phenyl)sulfonyl and (4-(quinolin-3-yloxy)phenyl)sulfonyl.

The term “heterocyclesulfonyl,” as used herein, refers to a heterocycle group, as defined herein, appended to the parent molecular moiety through a sulfonyl group, as defined herein. Representative examples of heterocyclesulfonyl include, but are not limited to, (pyridin-3-yl)sulfonyl and (quinolin-8-yl)sulfonyl.

The term “heterocyclethio,” as used herein, refers to a heterocycle group, as defined herein, appended to the parent molecular moiety through a thio moiety, as defined herein. Representative examples of heterocyclethio include, but are not limited to, pyrid-3-ylsulfanyl and quinolin-3-ylsulfanyl.

The term “heterocyclethioalkyl,” as used herein, refers to a heterocyclethio group, as defined herein, appended to the parent molecular moiety through an alkyl group, as defined herein. Representative examples of heterocyclethioalkyl include, but are not limited to, pyrid-3-ylsulfanylmethyl, (4-methylpyrimidin-2-yl)sulfanylmethyl, and 2-(quinolin-3-ylsulfanyl)ethyl.

The term “heterocyclethioalkylcarbonyl,” as used herein, refers to a heterocyclethioalkyl group, as defined herein, appended to the parent molecular moiety through a carbonyl group, as defined herein. Representative examples of heterocyclethioalkylcarbonyl include, but are not limited to, (pyrid-3-ylsulfanyl)acetyl, ((4-methylpyrimidin-2-yl)sulfanyl)acetyl, and (quinolin-3-ylsulfanyl)acetyl.

The term “hydroxy,” as used herein, refers to an —OH group.

The term “hydroxyalkyl,” as used herein, refers to one or two hydroxy groups, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypropyl and 2-ethyl-4-hydroxyheptyl.

The term “hydroxyalkylene” means an alkylene, as defined herein, containing 1 or hydroxy groups. Representative examples of hydroxyalkylene include, but are not limited to, —CH₂CH(OH)—, —CH₂CH(OH)CH₂—, —CH₂CH₂CH(OH)—, and —CH₂CH(OH)CH(OH)—.

The term “lower alkyl,” as used herein, is a subset of alkyl as defined herein and refers to a straight or a branched chain hydrocarbon group containing from 1 to 4 carbon atoms. Examples of lower alkyl are methyl, ethyl, n-propyl, iso-propyl, n-butyl, iso-butyl, sec-butyl, and tert-butyl.

The term “mercapto,” as used herein, refers to a —SH group.

The term “nitro,” as used herein, refers to a —NO₂ group.

The term “—NR_{A}R_{B},” as used herein, refers to two groups, R_{A} and R_{B}, which are appended to the parent molecular moiety through a nitrogen atom. R_{A} and R_{B} are each independently selected from hydrogen, alkyl, alkylcarbonyl and formyl. Representative examples of —NR_{A}R_{B} include, but are not limited to, acetylamino, amino, formylamino, dimethylamino and methylamino.

The term “(NR_{A}R_{B})alkyl,” as used herein, refers to a —NR_{A}R_{B} group, as defined herein, appended to the parent molecular moiety through an alkyl group, as defined herein. Representative examples of (NR_{A}R_{B})alkyl include, but are not limited to, (amino)methyl, (dimethylamino)methyl and (ethylamino)methyl.

The term “(NRARB)carbonyl,” as used herein, refers to a —NR_{A}R_{B} group, as defined herein, appended to the parent molecular moiety through a carbonyl group, as defined herein. Representative examples of (NR_{A}R_{B})carbonyl include, but are not limited to, aminocarbonyl, dimethylaminocarbonyl and ethylaminocarbonyl.

The term “(NR_{A}R_{B})sulfonyl,” as used herein, refers to an amino group, as defined herein, appended to the parent molecular moiety through a sulfonyl group, as defined herein. Representative examples of aminosulfonyl include, but are not limited to, aminosulfonyl, dimethylaminosulfonyl and ethylaminosulfonyl.

The term “oxo,” as used herein, refers to a ═O moiety.

The term “oxy,” as used herein, refers to a —O— moiety.

The term “phosphono,” as used herein, refers to a —P(O)(OR_{D})₂ group wherein R_{D} is selected from hydrogen and alkyl, as defined herein. Representative examples of phosphono include, but are not limited to, dimethoxyphosphoryl and diethoxyphosphoryl.

The term “sulfinyl,” as used herein, refers to a —S(O)— group.

The term “sulfono,” as used herein, refers to a —S(O)₂(OR_{E}) group wherein R_{E} is selected from alkyl, aryl, and arylalkyl, as defined herein. Representative examples of sulfono include, but are not limited to, methoxysulfonyl, ethoxysulfonyl, (benzyloxy)sulfonyl and phenoxysulfonyl.

The term “sulfonyl,” as used herein, refers to a —SO₂— group.

The term “thio,” as used herein, refers to a —S— moiety.

Method of the Invention

Compounds for the method of the invention, can be administered to modulate the activity of the histamine-3 receptors. Compounds for the method have an affinity for the histamine-3 receptors. In accordance with the method of the invention, the compounds can be administered to a human or animal for treatment and prevention of diseases or conditions related to histamine-3 receptors, for example diabetes and diabetes related diseases. The method of invention can be used for the treatment and prevention of diseases or conditions such as type II diabetes, insulin resistance syndrome, metabolic syndrome, Syndrome X, associated diseases, polycystic ovary syndrome, and other associated disorders.

The effects of example compounds have been demonstrated in various tests, including Examples A and B below.

### EXAMPLE A

### The Effect of 4′-{3-[(3R)-3-(Dimethylamino)pyrrolidinyl]propoxy}[1,1′-biphenyl]-4-carbonitrile on Insulin Tolerance

Assessment of the ability of H₃ receptor antagonists to ameliorate symptoms of diabetes has been determined in several ways. In the first instance, effects of an H₃ antagonist on the insulin tolerance test in mice fed a high-fat diet were determined. C57BL-6J mice (aged 5-6 weeks) were obtained from Jackson Labs (Bar Harbor, Me., U.S.A.) and individually housed at Abbott facilities under conditions of 12 h lights on, 12 h lights off (on at 22:00), with food and water available ad libitum. At the beginning of the study, mice were administered a standard diet (D12450B) or a high-fat content diet (D12451), both obtained from Research Diets Inc. (New Brunswick, N.J., U.S.A.) for approximately 14 weeks. Nine days prior to drug treatment, postprandial blood glucose was determined via a Medisense-G glucometer (Abbott Laboratories, Medisense Division, Bedford, Mass., U.S.A.). This was repeated at days 14 and 26 of drug treatment. On day 21, animals were fasted for 3 hours and fasting blood glucose determined in a blood sample obtained by tail snip. Insulin tolerance was determined by administering insulin (Lilly Humulin-R, 0.25 U/kg, i.p., obtained from Eli Lilly and Company, Indianapolis, Ind., U.S.A.) with blood glucose measured at 30, 60, 90 and 120 minutes using the glucometer. The insulin tolerance tests allowed for the specific evaluation of whole body insulin sensitivity. Pharmacological treatments were administered daily at 09:00 and 16:00. The 4′-{3-[(3R)-3-(dimethylamino)pyrrolidinyl]propoxy} [1,1′-biphenyl]-4-carbonitrile compound was administered p.o. at doses of 0.5, 5, and 15 mg/kg b.i.d., and dexfenfluramine at a dose of 10 mg/kg p.o., b.i.d. Data were analyzed using GraphPad InStat® (San Diego, Calif., U.S.A.) software using a one-way ANOVA software followed by Dunnett's post hoc test.

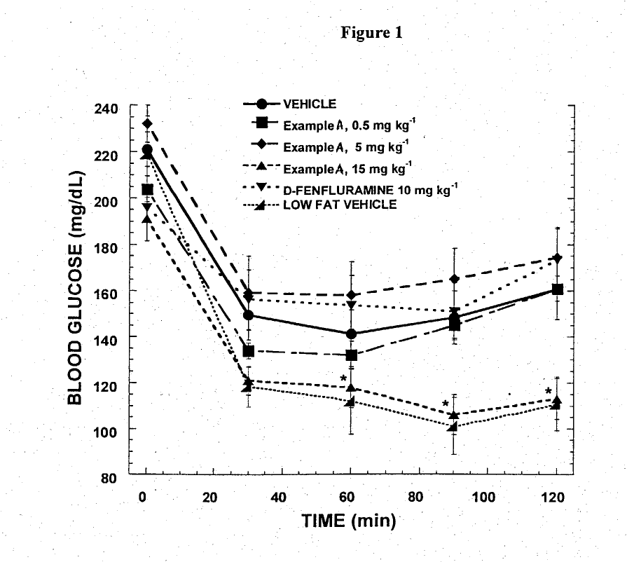

The results are shown in FIG. 1. Analysis of the results of this experiment showed that treatment with the compound of Example 2, Compound A, resulted in a dose-dependent improvement of the oral glucose tolerance test as shown in the figure below. The dose of 15 mg/kg p.o., administered twice daily completely normalized the oral glucose tolerance test, consistent with a potential improvement in the diabetic state of patients resistant to the glucose-lowering effects of insulin in type II diabetes.

### EXAMPLE B

### The Effect of 4′-[3-(3-Dimethylamino-pyrrolidin-1-yl)propoxyl-3′,5′-difluoro-biphenyl]-4-carbonitrile on Serum Triglyceride Levels

The effect of H₃ receptor blockade on serum triglyceride levels was ascertained. Elevated serum triglycerides typically are a marker of fatty acid spillover from adipose to non-adipose tissue as a result of positive net energy balance, increasing adipocyte triglyceride stores and insulin resistance, all characteristic of type II diabetes (for review see Lewis, G. F., et al., Disordered fat storage and mobilization in the pathogenesis of insulin resistance and type 2 diabetes, Endocrine Reviews, 23: 201-229, 2002.) 4′-[3-(3-Dimethylamino-pyrrolidin-1-yl)propoxy]-3′,5′-difluoro-biphenyl]-4-carbonitrile, prepared according to the procedures described in U.S. Publication 2002-0137931-A1, Example 172, was administered to mice treated in a similar manner to those in the study described above. C57BL-6J mice (aged -5-6 weeks) from Jackson Labs (Bar Harbor, Me., U.S.A.) were individually housed at Abbott facilities under conditions of 12 h lights on, 12 h lights off (on at 22:00), with food and water available ad libitum. At the beginning of the study, mice were administered a standard diet (D12450Bi) or a high-fat content diet (D12492i), both obtained from Research Diets Inc. (New Brunswick, N.J., U.S.A.) for approximately 16 weeks. Pharmacological treatments were administered daily at 09:00 and 16:00. 4′-[3-(3-Dimethylamino-pyrrolidin-1-yl)propoxy]-3′,5′-difluoro-biphenyl]-4-carbonitrile was administered p.o. at doses of 1, 3 and 10 mg/kg b.i.d., and sibutramine at a dose of 10 mg/kg p.o., b.i.d. Postprandial blood samples were drawn after 13 days of treatment. Mice were anesthetized with CO₂ gas, blood was obtained via cardiac puncture collected into tubes containing EDTA anticoagulant, and centrifuged to prepare plasma. Triglyceride concentration was determined spectrophotometrically, using a calorimetric assay kit (Sigma Chemical Co, St. Louis, Mo., U.S.A.). Data were analyzed using GraphPad InStat® (San Diego, Calif., U.S.A.) using a one-way ANOVA software followed by Dunnett's post hoc test.

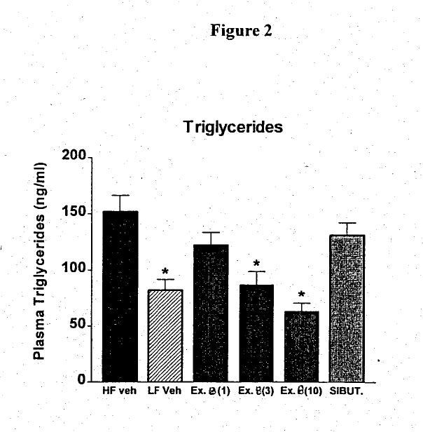

The results are shown in FIG. 2. 4′-[3-(3-Dimethylamino-pyrrolidin-1-yl)propoxy]-3′,5′-difluoro-biphenyl]-4-carbonitrile, Compound B, showed a clear, dose-dependent decrease in triglyceride levels. At a dose of 10 mg/kg, p.o. b.i.d., the triglyceride levels were reduced to the same level as those seen in low-fat diet fed mice, and the effects of 4′-[3-(3-Dimethylamino-pyrrolidin-1-yl)propoxy]-3′,5′-difluoro-biphenyl]-4-carbonitrile were not seen with the anti-obesity agent sibutramine, given to mice at 5 mg/kg, p.o. b.i.d.

Accordingly, an H₃ receptor ligand, for example, such as an H₃ receptor antagonist, can provide a useful composition for the prevention and/or treatment of conditions related to insulin resistance and adipocyte triglyceride stores, for example, insulin resistance syndrome, metabolic syndrome, Syndrome X, associated diseases, polycystic ovary syndrome, and other associated disorders.

Compounds suitable for the method can be administered in the form of a pharmaceutical composition. To prepare a suitable composition, a desired compound suitable for the method of the invention can be formulated with a pharmaceutically acceptable carrier. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compositions can be used for oral administration in solid or liquid form, either as an active agent or as the prodrug or metabolite of an active agent. Pharmaceutical compositions suitable for administration comprise one or more H₃ receptor agonists, antagonists, partial agonists, or inverse agonists, including salts or esters thereof, prepared and formulated in combination with one or more non-toxic pharmaceutically acceptable excipients.

Pharmaceutical compositions for the invention can be administered to humans and other mammals orally, sublingually, rectally, parenterally , intracisternally, intraurethrally, intravaginally, intraperitoneally, topically (as by powders, ointments or drops), bucally or as an oral or nasal spray. The term “parenterally,” as used herein, refers to modes of administration, for example intravenous, intramuscular, intraperitoneal, subcutaneous, and intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

Compositions also can contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. Such compositions also can include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, a desired compound can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Compounds for the method also can be in micro-encapsulated form, if appropriate, with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms an active compound can be admixed with at least one inert diluent such as sucrose, lactose, or starch. Such dosage forms also can comprise additional substances other than inert diluents, e.g., tableting lubricants and other tableting aids such a magnesium stearate and microcrystalline cellulose. In the case of capsules, tablets and pills, the dosage forms may also comprise buffering agents. Such dosage forms may optionally contain opacifying agents and can also be of such composition that they release an active compound only, or preferentially, in a certain part of the intestinal tract in a delayed manner.

Examples of embedding compositions which can be used include polymeric substances and waxes.

Injectable depot forms are made by forming microencapsulated matrices of the desired compound in biodegradable polymers such as polylactide-polyglycolide. Depending upon the ratio of the active agent to polymer and the nature of the particular polymer employed, the rate of release of the active agent can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the desired compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate;) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed in the method of the invention as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release an active compound only, or preferentially, in a certain part of the intestinal tract in a delayed manner. Examples of embedding compositions which can be used include polymeric substances and waxes.

Liquid dosage forms for oral administration include pharmaceutically acceptable emulsions, microemulsions, solutions, suspensions, syrups and elixirs. In addition to the active agent, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels also may contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the active agent, excipients such as lactose, talc, silicic acid, aluminum hydroxide, calcium silicates and polyamide powder, or mixtures of these substances. Sprays can additionally contain customary propellants such as chlorofluorohydrocarbons.

Any compound for the invention can be administered as a pharmaceutically acceptable salt derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66:1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate.

In addition, the method can be accomplished by administering a pharmaceutically acceptable ester of a desired compound. The term “pharmaceutically acceptable ester,” as used herein refers to esters of the desired compounds which hydrolyze in vivo and include those that break down readily in the human body to leave the parent compound or a salt thereof. Examples of pharmaceutically acceptable, non-toxic esters include, but are not limited to, C₁-to-C₆ alkyl esters and C₅-to-C₇ cycloalkyl esters, although C₁-to-C₄ alkyl esters are preferred. Such esters may be prepared according to suitable conventional methods.

The administration of the compounds as amides also is suitable for the method of the invention. In such case, the compound is administered as a pharmaceutically acceptable amide which, as used herein, refers to non-toxic amides of the desired compounds derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-toC₃ alkyl primary amides, and C₁-to-C₂ dialkyl secondary amides are preferred. Amides of the compounds may be prepared according to suitable conventional methods.

Compounds for the method of the invention also can be effective as a pharmaceutically acceptable prodrug. The term “pharmaceutically acceptable prodrug” or “prodrug,” as used herein, represents those prodrugs of the active compounds which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs may be transformed in vivo, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

Actual dosage levels of the compounds can be varied so as to obtain an amount to achieve the desired therapeutic response for a particular patient. The selected dosage level will depend upon the activity of the particular compound, the route of administration, the severity of the condition being treated and the condition and prior medical history of the patient being treated. It is within the purview of those with skill in the art to start doses of the active agent at levels lower than required to achieve the desired therapeutic effect and to gradually increase the dosage until the desired effect is achieved.

In particular, the method of the invention contemplates administering 4-(2-{2-[(2R)-2-methylpyrrolidinyl]ethyl}-1-benzofuran-5-yl)benzonitrile or 4-{2-[2-(2-methyl)-1-pyrrolidinyl)ethyl]-1-benzofuran-5-yl}benzonitrile obtained from either chemical synthesis or formed by in vivo biotransformation.

The term “therapeutically effective amount” of the compound of the invention means a sufficient amount of active compound to treat the disorder at a reasonable benefit/risk ratio applicable to any medical treatment. It will be understood, however, that the total daily usage of any compound for the method will be decided by the attending physician within the scope of sound medical judgment. The specific therapeutically effective dose level for any particular patient will depend upon a variety of factors including the disorder being treated; the active agent employed; the specific composition employed; the age, body weight, general health, sex and diet of the patient; the time of administration, route of administration, and rate of excretion of the active agent, the duration of the treatment; drugs used in combination or coincidental with the active agent; and like factors well known in the medical arts. For example, it is well within the skill of the art to start doses of an agonist at levels lower than required to achieve the desired therapeutic effect and to gradually increase the dosage until the desired effect is achieved.

The total daily dose of benzofuran and benzopyran derivatives administered to a human or lower animal may range from about 0.003 to about 10 mg/kg/day. Aminoalkoxybiphenylcarboxamide compounds may be administered in a range from about 0.003 to about 30 mg/kg/day. For purposes of oral administration, more preferable doses can be in the range of from about 0.01 to about 10 mg/kg/day. If desired, the effective daily dose can be divided into multiple doses for purposes of administration; consequently, single dose compositions may contain such amounts or submultiples thereof to make up the daily dose.

The foregoing is merely illustrative of the invention and is not intended to limit the scope of the invention, which is defined by the appended claims and any equivalents. Various modifications will be clear to one with skill in the art without departing from the spirit and scope of the invention.

What is claimed is:

1. A method of treating diabetic condition comprising administering to a patient in need of such treatment a therapeutically effective amount of a compound selected from the group consisting of:
a compound of formula (I): 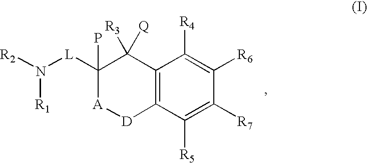
a compound of formula (III): 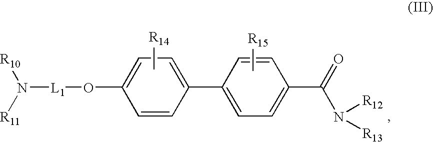
and a compound of formula (IV): 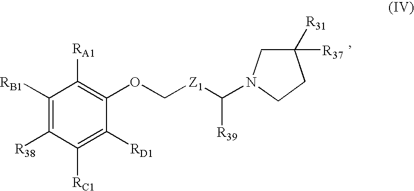
or pharmaceutically acceptable salts, esters, amides, or prodrugs thereof, wherein
A is selected from the group consisting of carbonyl and a covalent bond;
D is selected from the group consisting of O and S;
L is selected from the group consisting of lower alkylene, fluoroalkylene, and hydroxyalkylene;
P and Q taken together form a covalent bond or are both hydrogen;
R₁ and R₂ are each independently selected from the group consisting of hydrogen, alkyl, aryl, arylalkyl, cycloalkyl, cycloalkylalkyl, heterocycle, heterocyclealkyl, hydroxyalkyl, alkenyl, and alkynyl; or
R₁ and R₂ taken together with the nitrogen atom to which they are attached, together form a heterocycle;
R₃ is selected from the group consisting of hydrogen, alkoxy, alkoxycarbonyl, alkyl, alkylcarbonyl, alkylcarbonyloxy, alkylsulfinyl, alkylsulfonyl, alkylthio, aryl, carboxy, carboxyalkyl, cyano, cyanoalkyl, formyl, halogen, haloalkoxy, haloalkyl, heterocycle, hydroxy, hydroxyalkyl, mercapto, nitro, —NR_{A}R_{B}, (NR_{A}R_{B})alkyl, (NR_{A}R_{B})carbonyl, and (NR_{A}R_{B})sulfonyl;
R₄, R₅, R₆ and R₇ are each independently selected from the group consisting of hydrogen, alkoxy, alkoxycarbonyl, alkyl, alkylcarbonyl, alkylcarbonyloxy, alkylsulfinyl, alkylsulfonyl, alkylthio, aryl, carboxy, carboxyalkyl, cyano, cyanoalkyl, cycloalkyl, formyl, halogen, haloalkoxy, haloalkyl, heterocycle, hydroxy, hydroxyalkyl, mercapto, nitro, —NR_{A}R_{B}, (NR_{A}R_{B})alkyl, (NR_{A}R_{B})carbonyl, (NR_{A}R_{B})sulfonyl, -L₂R₂₀, and —R₂₀L₃R₂₂, provided that at least one of R₄, R₅, R₆, or R₇ is aryl, heterocycle, cycloalkyl, -L₂R₂₀ or —R₂₀L₃R₂₂;
L₂ is selected from the group consisting of alkylene, alkenylene, O, S, S(O), S(O)₂, C(═O), C=(NOR₂₁), and N(R_{A});
L₃ is selected from the group consisting of a covalent bond, alkylene, alkenylene, O, S, C(═O), N(═OR₂,), and N(R_{A});
R₁₀ and R₁₁ are each independently selected from the group consisting of hydrogen, alkyl, aryl, arylalkyl, cycloalkyl, cycloalkylalkyl, heterocycle and heterocyclealkyl; or
R₁₀ and R₁₁ taken together with the nitrogen atom to which they are attached, together form a heterocycle selected from the group consisting of azepanyl, azetidinyl, morpholinyl, piperazinyl, piperidinyl, pyrrolidinyl, 2,5-dihydro-1H-pyrrolyl, pyrrolyl, thiomorpholinyl and 1,1-dioxidothiomorpholinyl, provided that when R₁₀ and R₁₁ together form pyrrolidinyl and wherein said pyrrolidinyl is substituted with 1 substituent then said substituent is other than alkoxy, hydroxy or —NR_{A}R_{B};
R₁₂ and R₁₃ are each independently selected from the group consisting of hydrogen, alkyl, aryl, arylalkyl, cycloalkyl, cycloalkylalkyl, heterocycle and heterocyclealkyl; or
R₁₂ and R₁₃ taken together with the nitrogen atom to which they are attached, together form a heterocycle selected from the group consisting of azepanyl, azetidinyl, morpholinyl, piperazinyl, piperidinyl, pyrrolidinyl, 2,5-dihydro-1H-pyrrolyl, pyrrolyl, thiomorpholinyl and 1,1-dioxidothiomorpholinyl;
R₁₄ and R₁₅ are each independently selected from the group consisting of hydrogen, alkenyl, alkoxy, alkoxyalkoxy, alkoxyalkyl, alkoxycarbonyl, alkyl, alkylcarbonyl, alkylcarbonyloxy, alkylsulfinyl, alkylsulfonyl, alkylthio, alkynyl, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
R₂₀ is selected from the group consisting of aryl, heterocycle, and cycloalkyl;
R₂₁ is selected from the group consisting of hydrogen and alkyl;
R₂₂ is selected from the group consisting of aryl, heterocycle, and cycloalkyl;
R_{A} and R_{B} are each independently selected from hydrogen, alkyl, alkylcarbonyl or formyl;
Z₁ is selected from the group consisting of a covalent bond and CH₂;
R₃₁ is selected from the group consisting of OR₃₂, NR₃₃R₃₄ and 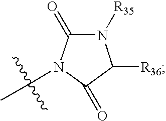
R₃₂ is selected from the group consisting of hydrogen, alkoxycarbonyl, alkyl, alkylcarbonyl, aminocarbonyl, sulfono and phosphono;
R₃₃ and R₃₄ are independently selected from the group consisting of hydrogen, alkenyl, alkenylcarbonyl, alkenyloxycarbonyl, alkenylsulfonyl, alkoxycarbonyl, alkyl, alkylcarbonyl, alkylsulfonyl, alkynyl, alkynylcarbonyl, alkynyloxycarbonyl, alkynylsulfonyl, aminocarbonyl, aminosulfonyl, arylalkyl, arylalkenylcarbonyl, arylalkenylsulfonyl, arylalkylcarbonyl, arylalkylsulfonyl, arylarylcarbonyl, arylarylsulfonyl, arylcarbonyl, arylheterocylecarbonyl, arylheterocylesulfonyl, aryloxyarylcarbonyl, aryloxyarylsulfonyl, arylsulfonyl, cycloalkyl, cycloalkylalkyl, cycloalkylalkylcarbonyl, cycloalkylalkylsulfonyl, cycloalkylcarbonyl, cycloalkylsulfonyl, formyl, heterocycle, heterocyclealkyl, heterocyclealkylcarbonyl, heterocyclealkylsulfonyl, heterocyclearylcarbonyl, heterocyclearylsulfonyl, heterocyclecarbonyl, heterocycleheterocyclecarbonyl, heterocycleheterocyclesulfonyl, heterocycleoxyalkylcarbonyl, heterocycleoxyarylcarbonyl, heterocycleoxyarylsulfonyl, heterocyclesulfonyl, and heterocyclethioalkylcarbonyl;
R₃₅ and R₃₆ are independently selected from the group consisting of hydrogen and alkyl;
R₃₇ is selected from the group consisting of hydrogen and alkyl; or
R₃₁ and R₃₇ together form (═O);
R₃₈ is selected from the group consisting of alkylcarbonyl, aryl, arylcarbonyl, arylcarbonylaryl, arylcarbonylheterocycle, cycloalkylcarbonyl, cycloalkylcarbonylaryl, cycloalkylcarbonylheterocycle, heterocycle, heterocyclecarbonyl, heterocyclecarbonylaryl, and heterocyclecarbonylheterocycle;
R₃₉ is selected from the group consisting of hydrogen and lower alkyl; and
R_{A1}, R_{B1}, R_{C1} and R_{D1} are independently selected from the group consisting of hydrogen, alkenyl, alkoxy, alkoxyalkoxy, alkoxyalkyl, alkoxycarbonyl, alkyl, alkylcarbonyl, alkylcarbonyloxy, alkylsulfinyl, alkylsulfonyl, alkylthio, alkynyl, amino, aminoalkyl, aminocarbonyl, carboxy, carboxyalkyl, cyano, cyanoalkyl, formyl, halogen, haloalkoxy, haloalkyl, hydroxy, hydroxyalkyl, mercapto or nitro.

2. The method of claim 1 wherein the compound has the formula (II): 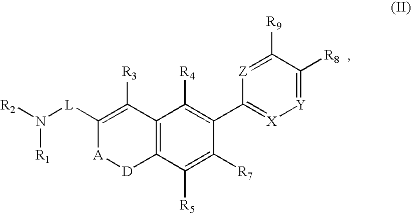
or pharmaceutically acceptable salts, esters, amides, or prodrugs thereof, wherein
R₇ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;
R₈ is selected from hydrogen, alkylcarbonyl, arylcarbonyl, cyano, cycloalkylcarbonyl, heterocyclecarbonyl or (NR_{A}R_{B})carbonyl;
R₉ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;
X is selected from CH, CR_{X} or N;
Y is selected from CH, CR_{Y} or N;
Z is selected from CH, CR_{Z} or N;
R_{X}, R_{Y} and R_{Z} groups are each independently selected from alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl.

3. The method of claim 1 wherein the compound is selected from the group consisting of 4-(2-{2-[(2R)-2-methylpyrrolidinyl]ethyl}-1-benzofuran-5-yl)benzonitrile and 4-{2-[2-(2-methyl)-1-pyrrolidinyl)ethyl]-1-benzofuran-5-yl}benzonitrile.

4. The method of claim 1 wherein the compound is 4′-{3-[(3R)-3-(dimethylamino)pyrrolidinyl]propoxy}[1,1′-biphenyl]-4-carbonitrile and 4′-[3-(3-dimethylamino-pyrrolidin-1-yl)propoxy]-3′,5′-difluoro-biphenyl]-4-carbonitrile.

5. The method of claim 1 wherein the diabetic condition is selected from the group consisting of type II diabetes, insulin resistance syndrome, metabolic syndrome, Syndrome X, and polycystic ovary syndrome.

6. The method of claim 1 wherein the diabetic condition is type II diabetes.

7. The method of claim 1 wherein the patient is a human or animal.

8. The method of claim 1 wherein the compound of formula (I) is administered in an amount of from about 0.003 mg/kg/day to about 10 mg/kg/day.

9. The method of claim 1 wherein the compound of formula (III) is administered in an amount of from about 0.003 mg/kg/day to about 30 mg/kg/day.

10. The method of claim 1 wherein the compound of formula (III) is administered in an amount of from about 0.01 mg/kg/day to about 10 mg/kg/day.

11. A method of treating a diabetic condition comprising administering to a patient in need of such treatment a therapeutically effective amount of a compound having H₃ receptor activity.
